(19) United States
(12) Patent Application Publication
(10) Pub. No.: US 2026/0237505 A1
(43) Pub. Date: Aug. 13, 2026

(54) INFORMATION CONVERSION SYSTEM AND BIOLOGICAL INFORMATION DETECTION APPARATUS

(71) Applicant: CANON KABUSHIKI KAISHA, Tokyo (JP)

(72) Inventors: SOMA NAKAI, Tokyo (JP); TAIHEI MUKAIDE, Kanagawa (JP); CHIHIRO NAGURA, Tochigi (JP); TAKESHI KONDOH, Tokyo (JP); MASAHIRO NAKANISHI, Kanagawa (JP)

(21) Appl. No.: 19/637,536

(22) Filed: Apr. 2, 2026

Related U.S. Application Data
(63) Continuation of application No. PCT/JP2024/034977, filed on Sep. 30, 2024.

(30) Foreign Application Priority Data
Oct. 6, 2023 (JP) 2023-174306
Apr. 23, 2024 (JP) 2024-070111

(51) Int. Cl. G16H 40/63

(52) U.S. Cl. CPC G16H 40/63

(57) ABSTRACT

The present disclosure provides an information conversion system or a biological information detection apparatus including a sensor that can be repeatedly worn. An information conversion system according to the present disclosure outputs character information or audio information converted from biological information about a user. The information conversion system includes a first member to be fixed to the user, a biological information detection unit configured to bring a contact member into contact with the user to detect biological information, and a second member placed on the first member and configured to bias the biological information detection unit in a direction in which the biological information detection unit contacts the user, wherein the biological information detection unit includes a posture control member configured to control a posture of the contact member.

### CROSS-REFERENCE TO RELATED APPLICATIONS

This application is a Continuation of International Patent Application No. PCT/JP2024/034977, filed September 30, 2024, which claims the benefit of Japanese Patent Applications No. 2023-174306, filed October 6, 2023, and No. 2024-070111, filed April 23, 2024, all of which are hereby incorporated by reference herein in their entirety.

### BACKGROUND

### Field of the Technology

The present disclosure relates to an information conversion system and a biological information detection apparatus that convert biological information into character information.

### DESCRIPTION OF THE RELATED ART

A technique for recognizing the content of the utterance of a user using audio information about the user has recently been used. A method for detecting biological signals from the skin surface of a human body and estimating states of jaw and oral movements is described (e.g., Japanese Patent No. 5924724).

According to Japanese Patent No. 5924724, a sensor (surface electrode) for detecting biological information is placed on a submental area with a film sheet. Accordingly, it may be difficult to repeatedly wear the sensor due to deterioration in the adhesion of the film sheet or the like.

### SUMMARY

In view of the above, the present disclosure is directed to providing an information conversion system or a biological information detection apparatus including a sensor that can be repeatedly worn.

However, the issues that the embodiments disclosed in this specification and drawings attempt to solve are not limited to the above-described issues. Issues corresponding to the effects of each configuration described in the following embodiments can also be treated as other issues.

To achieve an object of the present disclosure, an information conversion system that outputs character information or audio information converted from biological information about a user includes a first member to be fixed to the user, a biological information detection unit configured to bring a contact member into contact with the user to detect biological information, and a second member placed on the first member and configured to bias the biological information detection unit in a direction in which the biological information detection unit contacts the user. The biological information detection unit includes a posture control member configured to control a posture of the contact member.

A biological information detection apparatus includes a first member to be fixed to a user, a biological information detection unit configured to bring a contact member into contact with the user to detect biological information, and a second member placed on the first member and configured to bias the biological information detection unit in a direction in which the biological information detection unit contacts the user. The biological information detection unit includes a posture control member configured to control a posture of the contact member.

Features of the present disclosure will become apparent from the following description of embodiments with reference to the attached drawings.

### BRIEF DESCRIPTION OF THE DRAWINGS

FIG. 1 is a block diagram illustrating a configuration of an information conversion system according to the present disclosure.

FIG. 2 illustrates an appearance of the information conversion system according to the present disclosure.

FIG. 3 illustrates a configuration of a biological information detection unit according to the present disclosure.

FIG. 4 illustrates a configuration of a biological information detection unit according to the present disclosure.

FIG. 5 illustrates an embodiment of a biological information detection unit according to the present disclosure.

FIG. 6 illustrates an embodiment of a biological information detection unit according to the present disclosure.

FIG. 7 illustrates a configuration of a biological information detection unit according to the present disclosure.

FIG. 8 illustrates an embodiment of a biological information detection unit according to the present disclosure.

FIG. 9 illustrates an embodiment of a biological information detection unit according to the present disclosure.

FIG. 10 illustrates a configuration of a biological information detection unit according to Modified Example 1.

FIG. 11 illustrates a configuration of a biological information detection unit according to Modified Example 2.

FIG. 12 illustrates a state where a displacement of a light-emitting element according to Modified Example 2 is detected.

FIG. 13 illustrates a state where a displacement of the light-emitting element according to Modified Example 2 is detected.

FIG. 14 illustrates an appearance of an information conversion system according to Modified Example 3.

FIG. 15 illustrates an appearance of an information conversion system according to Modified Example 4.

FIG. 16 illustrates an appearance of an information conversion system according to Modified Example 5.

FIG. 17 illustrates an appearance of an information conversion system according to Modified Example 6.

FIG. 18 illustrates an appearance of an information conversion system according to Modified Example 7.

FIG. 19 illustrates an appearance of an information conversion system according to Modified Example 8.

FIG. 20 illustrates an appearance of an information conversion system according to Modified Example 9.

FIG. 21 illustrates an appearance of an information conversion system according to Modified Example 10.

### DESCRIPTION OF THE EMBODIMENTS

Desirable embodiments of the present disclosure will be described below with reference to the drawings. The dimensions, materials, and shapes of components described in the following embodiments, the relative arrangement of the components, and the like should be appropriately modified in accordance with the configuration of an apparatus to which the disclosure is applied and various conditions. The scope of the disclosure is not intended to be limited to the following embodiments.

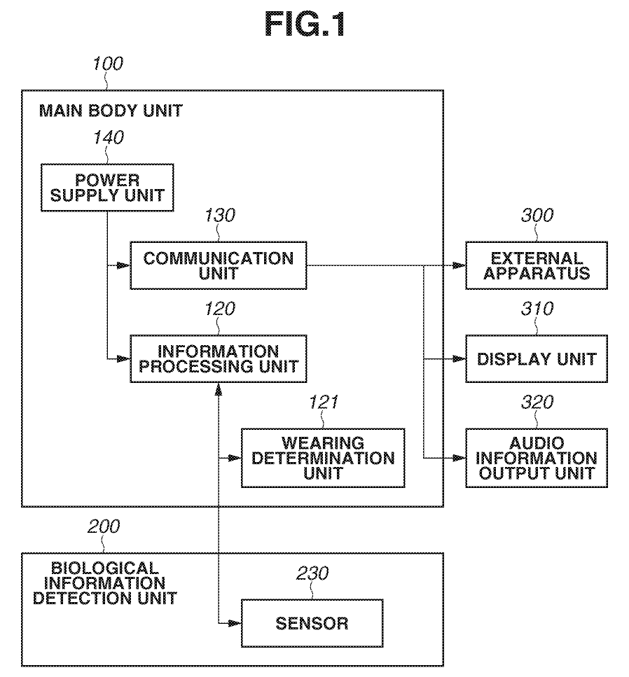

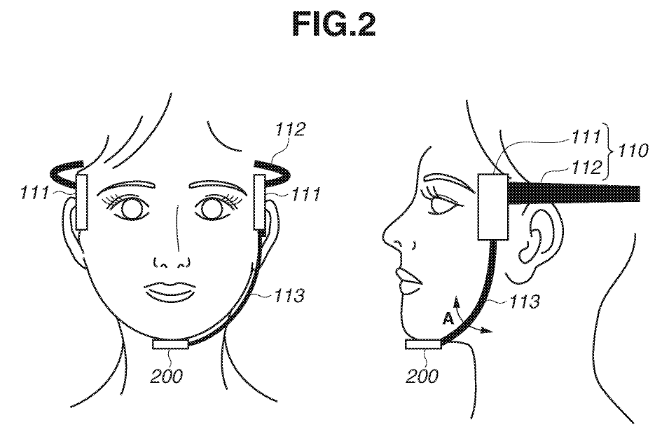

FIG. 1 is a block diagram illustrating a configuration of an information conversion system according to the present disclosure. FIG. 2 illustrates an appearance of the information conversion system.

The information conversion system according to an embodiment of the present disclosure mainly includes a main body unit 100 and a biological information detection unit 200. The information conversion system may include an external apparatus 300, a display unit 310, and an audio information output unit 320.

The external apparatus 300 has similar functions to, for example, an information processing unit 120, and is connected to the main body unit 100.

The display unit 310 has a function for displaying character information and the like, and is connected to the main body unit 100. The main body unit 100 may include a display control unit (not illustrated) for controlling a display form on the display unit 310.

The audio information output unit 320 is a speaker and is connected to the main body unit 100. The audio information output unit 320 is configured to reproduce audio information.

While FIGS. 1 and 2 illustrate a configuration including a single biological information detection unit 200, a plurality of biological information detection units 200 may be provided. The biological information detection unit 200 includes a sensor 230 for detecting biological information about movements of muscles, movements of skin, mouth, and tongue of a user, and the like. If a plurality of biological information detection units 200 is provided, the biological information detection units 200 detect biological information at different respective positions. The sensor 230 in each biological information detection unit 200 is at least one of a myopotential sensor, an acceleration sensor, an ultrasonic sensor, a tactile sensor, an optical sensor, a pressure sensor, and the like.

The myopotential sensor is a sensor for detecting electric signals generated when muscles are moved. For example, a tripolar Ag electrode is used. The electrode is placed in the vicinity of a belly muscle of a muscle linked to the mouth or tongue to be measured.

The acceleration sensor is a sensor for detecting an acceleration and outputting data or signals depending on the detected acceleration. The angular velocity sensor (gyroscope sensor) is a sensor for detecting an angular velocity and outputting data or signals depending on the detected angular velocity. The acceleration sensor and the angular velocity sensor in the biological information detection unit 200 may be integrated together. As a sampling frequency for biological information, for example, 400 Hz is set for the six-axis acceleration angular velocity sensors and, for example, 800 Hz is set for the myopotential sensor.

The biological information detection unit 200 is placed at a position where movements of the mouth and tongue of the user can be detected. The biological information detection unit 200 is placed in the vicinity of the mouth of the user. Specifically, the biological information detection unit 200 is placed on a submental region, a buccal region, or the like of the user. The biological information detection unit 200 may be placed in any region other than the above-described regions, as long as biological information about movements of the mouth and tongue of the user can be detected.

The main body unit 100 is a device that is fixed to the user through a frame member 110 (first member) and processes biological information obtained by the biological information detection unit 200. As illustrated in FIG. 1, the main body unit 100 mainly includes the information processing unit 120, a communication unit 130, and a power supply unit 140. Although not illustrated, the main body unit 100 is placed in the frame member 110 (first member) illustrated in FIG. 2.

The frame member 110 (first member) has a function for supporting the components of the main body unit 100 and sandwiching the head or neck of the user, thereby being fixed to the user.

As illustrated in FIG. 2, the frame member 110 (first member) is fixed to the user by two sandwiching portions 111 that sandwich both temples of the user and a connecting portion 112 that connects the two sandwiching portions 111.

The connecting portion 112 is a curved frame that goes around the back of the user's head and connects the two sandwiching portions 111. The connecting portion 112 is a frame having elasticity for sandwiching the user's head. In other words, the frame member 110 (first member) has such a shape that the frame member goes around the user's head and has elasticity for sandwiching the user's head. Accordingly, the two sandwiching portions 111 sandwich the user's head with a predetermined pressure due to the elasticity of the connecting portion 112.

Thus, from the viewpoint of wearability, it may be desirable to locate the connecting portion 112 such that the connecting portion goes around the region in the vicinity of the back of the user's head or the top of the user's head. Alternatively, the connecting portion may lie across the front side of the face, like a head band or glasses. The components of the main body unit 100 are incorporated in the sandwiching portions 111 of the frame member 110 (first member), but instead may be incorporated in a space formed in the connecting portion 112. Thus, the components of the main body unit 100 are incorporated in the sandwiching portions 111 or in the connecting portion 112.

The frame member 110 (first member) may have any configuration, as long as the frame member can be fixed to the user. For example, a choker type member to be worn around the neck of the user, or a face mask type member that covers the mouth of the user may be used. The frame member 110 (first member) may desirably include an adjustment mechanism so that various users can wear the frame member. As the adjustment mechanism, for example, the frame member 110 (first member) includes a slide mechanism (expansion/contraction mechanism).

The connecting portion 112 includes a plurality of tubular members to form the slide mechanism (expansion/contraction mechanism). Each of the plurality of tubular members is curved. Each of the plurality of tubular members is curved along, for example, the shape of the back of the user's head. A second tubular member is removably connected to a first tubular member that constitutes the plurality of tubular members. The length of the plurality of curved tubular members can be adjusted by inserting or removing the second tubular member into or from the first tubular member. Thus, the slide mechanism enables the connecting portion 112 to be expanded and contracted.

If the connecting portion 112 of the frame member 110 (first member) does not include the adjustment mechanism, the elasticity can be imparted to the connecting portion 112 so as to be adapted to individual users.

A biasing member 113 (second member) that biases the biological information detection unit 200 is placed on (extended from) the frame member 110 (first member). The biasing member 113 (second member) is a member that biases the biological information detection unit 200 to be described below in a direction in which the biological information detection unit contacts the user. The biasing member 113 (second member) has elasticity in a direction in which the biasing member contacts the user so as to maintain a biasing force even when the user is performing an operation. Thus, the biasing member 113 (second member) enables the biological information detection unit 200 to be pressed against the user.

The present embodiment includes the first member (frame member 110) to be fixed to the user and the biological information detection unit 200 that brings a contact member 210 into contact with the user to detect biological information. The present embodiment further includes the second member (biasing member 113) that is placed on (extended from) the first member (frame member 110) and biases the biological information detection unit 200 in the direction in which the biological information detection unit contacts the user.

The biasing member 113 (second member) is connected to the biological information detection unit 200. The biological information detection unit 200 is a component that moves along with the oral movement of the user. Accordingly, the biasing member 113 (second member) may have elasticity so that the biological information detection unit 200 can be pressed against the user and the biasing member can be deformed along with the oral movement. The rigidity of the biasing member 113 (second member) is set to a first predetermined value or higher so that the biological information detection unit 200 can be biased in the direction in which the biological information detection unit contacts the user. The biasing member 113 (second member) is configured to be deformed along with the movement of the user. On the other hand, the connecting portion 112 (frame member 110 (first member)) is configured not to be deformed along with the movement of the user. Accordingly, the rigidity of the biasing member 113 (second member) is lower than the rigidity of the connecting portion 112 (frame member 110 (first member)). The rigidity of the connecting portion 112 (frame member 110 (first member)) is set to a second predetermined value or higher. Thus, the first predetermined value for the rigidity of the biasing member 113 (second member) is lower than the second predetermined value for the rigidity of the connecting portion 112 (frame member 110 (first member)). The rigidity is at least one of bending rigidity and shear rigidity.

The biasing member 113 (second member) may be desirably configured to adjust the biasing force so as not to inhibit the oral movement of the user. Like the connecting portion 112, the biasing member 113 (second member) includes an adjustment mechanism, which enables adjustment of the biasing force of the biological information detection unit 200 connected to the biasing member 113 (second member). Examples of the adjustment mechanism include the slide mechanism (expansion/contraction mechanism) in the biasing member 113 (second member).

The biasing member 113 (second member) includes a plurality of tubular members to form the slide mechanism. Each of the plurality of tubular members is curved. Each of the plurality of tubular members is curved along, for example, the shape of the cheeks of the user so as not to inhibit the oral movement of the user. The second tubular member is removably connected to the first tubular member that constitutes the plurality of tubular members. The length of the plurality of curved tubular members can be adjusted by inserting or removing the second tubular member into or from the first tubular member. This configuration enables the biasing member 113 (second member) to be expanded and contracted.

Further, the biasing member 113 (second member) may desirably include a retracting mechanism or the like so that the biasing member can retract when the user puts on or takes off the device. The sandwiching portions 111 (frame member 110 (first member)) includes a rotation mechanism (not illustrated) that supports the biasing member 113 (second member) and rotates the biasing member 113 (second member). The rotation mechanism is connected to the biasing member 113 (second member) and has a function for rotating the biasing member 113 (second member) around the sandwiching portions 111. Since the biological information detection unit 200 is connected to the biasing member 113 (second member), the biasing member 113 (second member) can be moved in a direction "A" as illustrated in FIG. 2 by rotating the biasing member 113 (second member) using the rotation mechanism. In other words, the rotation mechanism enables adjustment of a wearing posture of the biological information detection unit 200 and an angle of the biological information detection unit 200.

The biasing member 113 (second member) may further incorporate a substrate and wiring for inputting and outputting power and signals between the main body unit 100 and the biological information detection unit 200. In other words, the biasing member 113 (second member) also functions as a conducting path for establishing an electric connection.

The information processing unit 120 is a device that processes the biological information obtained by the biological information detection unit 200 to be described below. However, the information processing unit 120 need not necessarily perform all the processes of converting the biological information into character information or audio information, and may process signals so that the signals can be transmitted to the external apparatus 300. The information processing unit 120 may be further provided with an input unit to receive a user operation, or may be operated from the external apparatus 300.

In the case of converting the biological information detected by the biological information detection unit 200 into character information or audio information, the information processing unit 120 converts the biological information into character information or audio information by a conversion technique. As a conversion algorithm in the conversion technique, a learned model based on an architecture constructed by a neural network is used. The information processing unit 120 includes a storage unit (not illustrated) that stores the learned model, and performs inference processing using the learned model.

The learned model is a model generated using Convolutional Neural Network (CNN) or Recurrent Neural Network (RNN) as deep learning. A model derived from the CNN or RNN may also be used. Not only the model derived from the CNN or RNN, but also any other machine learning technique such as a support vector machine, logistic regression, or random forest, and a rules-based technique may be used.

The information processing unit 120 generates the learned model to be used for the conversion technique by learning, for example, the biological information detected by the biological information detection unit 200 and character information or audio information in association with each other.

Specifically, the information processing unit 120 obtains a plurality of data sets in which the biological information detected by the biological information detection unit 200 is associated with character information or audio information (e.g., "a", "i", "u", "e", "o") or the sounds of these characters in advance.

The information processing unit 120 uses, as training data, the correspondence relation between the biological information and character information or audio information in the plurality of data sets. The learned model to be used in the conversion technique is generated by fine-tuning the original learned model using the training data. Thus, inference processing can be performed on newly input biological information using the learned model generated by learning the biological information and character information or audio information in association with each other, and character information or audio information can be output. As the conversion algorithm used in the conversion technique, different conversion algorithms may be used as a conversion algorithm for a silence period and a conversion algorithm for an utterance period.

A learning unit is configured in the information processing unit 120. The learning unit may be configured in the external apparatus 300 or in the cloud. The information processing unit 120 may include a storage unit that stores the learned model. The information processing unit may have any configuration, as long as learned models can be used on the information processing unit 120.

The learning unit obtains a plurality of data sets in which the biological information detected by the biological information detection unit 200 is associated with character information or audio information in advance. The learning unit generates a learned model by learning the biological information and character information or audio information in association with each other using the correspondence relation between the biological information and character information or audio information in the plurality of data sets as training data. Thus, the information processing unit 120 can perform inference processing on newly input biological information using the learned model generated by learning the biological information and the character information or audio information in association with each other, and can output character information or audio information.

The information processing unit 120 may also use different conversion algorithms (learned models) for the user as the conversion algorithm for the silence period and the conversion algorithm for the utterance period. The silence period is a period in which the user is not uttering or remains silent. The utterance period is a period in which the user is uttering.

The learning unit distinguishes the silence period from the utterance period, and generates a learned model for the silence period and a learned model for the utterance period. Specifically, the learning unit distinguishes the silence period from the utterance period, and generates the learned model for the silence period and the learned model for the utterance period by learning the biological information and character information or audio information in association with each other using the correspondence relation between the biological information and character information or audio information in the plurality of data sets as training data. The information processing unit 120 applies the learned model for the silence period during the silence period in which the user remains silent, and applies the learned model for the utterance period during the utterance period. The information processing unit 120 can perform inference processing on newly input biological information obtained during the silence period or newly input biological information obtained during the utterance period, and can output character information or audio information.

The external apparatus 300 may have the functions of the information processing unit. The biological information detection unit 200 transmits biological information detected by the sensor 230 to the external apparatus 300. The external apparatus 300 converts the biological information into character information or audio information, and transmits the converted character information or audio information to the communication unit 130. The display unit 310 displays the character information. The converted character information may be further converted into audio information to be transmitted to the audio information output unit 320. The external apparatus 300 may directly convert the biological information into audio information and may transmit the audio information to the audio information output unit 320.

The communication between the biological information detection unit 200 and the main body unit 100 may be wired communication or wireless communication. If the communication between the biological information detection unit 200 and the main body unit 100 is implemented by wired communication, the sensor 230 and the information processing unit 120 are connected with a wire such as a universal serial bus (USB) cable.

If the communication between the sensor 230 and the information processing unit 120 is implemented by wireless communication, a wireless connection is established by wireless local area network (LAN) communication such as Wi-Fi, short‑range wireless communication such as Bluetooth®, or the like.

The information processing unit 120 according to the present embodiment further includes a wearing determination unit 121. The wearing determination unit 121 is a device that determines whether the biological information detection unit 200 satisfies a wearing condition.

The wearing determination unit 121 determines whether the wearing condition for the biological information detection unit 200 (sensor 230) is satisfied based on whether the biological information detected by the biological information detection unit 200 (sensor 230) has been appropriately detected. The wearing determination unit 121 may be provided with another sensor. However, the use of biological information also makes it possible to determine whether the wearing condition is satisfied. During the determination, the user may be caused to perform a predetermined operation to determine the wearing condition.

Specifically, the wearing determination unit 121 determines whether the wearing condition is satisfied based on a signal intensity, signal characteristics, or the like in the biological information detected by the sensor 230. For example, if the signal intensity in the biological information detected by the sensor 230 is more than or equal to a predetermined value, the wearing determination unit 121 determines that the wearing condition is satisfied. If the signal intensity is less than the predetermined value, the wearing determination unit 121 determines that the wearing condition is not satisfied. If the signal characteristics in the biological information detected by the sensor 230 are close (similar) to predetermined characteristics, the wearing determination unit 121 determines that the wearing condition is satisfied. If the signal characteristics are not close (not similar) to the predetermined characteristics, the wearing determination unit 121 determines that the wearing condition is not satisfied. If the wearing determination unit 121 determines that the wearing condition is not satisfied, or the wearing state is not appropriate, the display unit 310 displays a determination result.

It may be desirable for the wearing determination unit 121 to constantly determine whether the wearing condition is satisfied. However, the determination may be made only at the time of wearing the device. If the wearing determination unit 121 determines that the wearing condition is not satisfied, a notification indicating that the wearing condition is not satisfied may be desirably issued to the user via the display unit 310 or the audio information output unit 320.

The communication unit 130 is a device that communicates signals processed by the information processing unit 120 with the external apparatus 300, the display unit 310, and the audio information output unit 320. As the external apparatus 300, a personal computer (PC), a smartphone, a tablet PC, or the like may be mainly used. However, the external apparatus 300 is not limited to these examples, and may be configured on the cloud. The communication unit 130 may establish wired communication or wireless communication. If the communication is implemented by wired communication, the communication unit 130 is connected to each of the external apparatus 300, the display unit 310, and the audio information output unit 320 with a wire such as a USB cable. If the communication is implemented by wireless communication, the communication unit 130 is wirelessly connected to each of the external apparatus 300, the display unit 310, and the audio information output unit 320 by wireless LAN communication such as Wi-Fi, short-range wireless communication such as Bluetooth®, or the like.

The power supply unit 140 is a device that supplies power to each of the information processing unit 120 and the communication unit 130. A secondary battery that can be repeatedly charged may be desirably used as the power supply unit 140. The main body unit 100 may include a microphone that receives the utterance of the user.

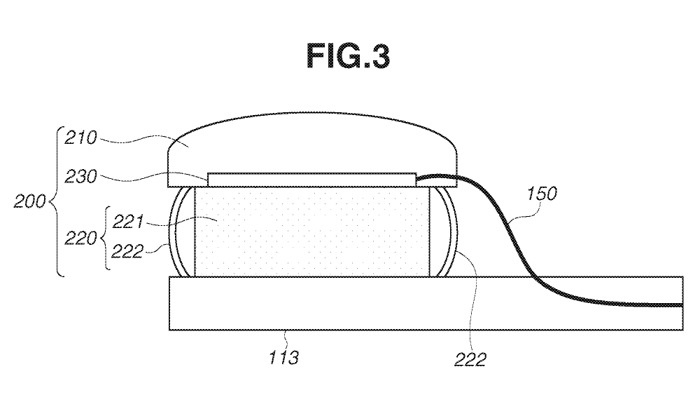

FIG. 3 illustrates a configuration of the biological information detection unit 200. The biological information detection unit 200 is a device that detects biological information from the skin surface of the user. The biological information detection unit 200 is placed at a leading end of the biasing member 113 (second member) that biases the biological information detection unit 200 in the direction in which the biological information detection unit contacts the user. In FIG. 3, the biological information detection unit 200 is brought into contact with the user and is biased in an upward direction. The biological information detection unit 200 is configured by mainly using the contact member 210, a posture control member 220, and the sensor 230. The biological information detection unit 200 may be desirably placed in a region such as a neck region, a submental region, a cheek region, or a temple region of the user so as to detect biological information about movements of the mouth and tongue of the user. The biological information detection unit 200 may be placed in any region other than the above-described regions, as long as biological information about movements of the mouth and tongue can be detected.

The sensor 230 is a member that detects biological information about movements of muscles, movements of the skin and tongue of the user, and the like. The sensor used for the biological information detection unit 200 is at least one of a myopotential sensor, an acceleration sensor, an ultrasonic sensor, a tactile sensor, an optical sensor, a pressure sensor, and the like. The sensor 230 is connected to a signal line 150. The signal line 150 transmits biological signals detected by the sensor 230. The biological signals obtained in the biological information detection unit 200 are transmitted to the information processing unit 120 of the main body unit 100 via the signal line 150.

The biological information detection unit 200 may be any sensor other than the above-described sensors, as long as the biological information detection unit can function as a sensor for detecting biological information. A combination of a plurality of sensors may also be used. The signals obtained by the sensor 230 are processed by the information processing unit 120 described above.

The contact member 210 is a member that contacts the skin surface of the user. The contact member 210 may desirably have a smooth surface so as to closely adhere to the skin of the user. The contact member 210 may have any configuration other than the above-described configuration depending on the detection method. For example, in the case of using a myopotential sensor as the sensor 230 of the biological information detection unit 200, the contact member 210 is provided with a plurality of electrodes. Accordingly, the contact member 210 may have irregularities so that the electrodes of the myopotential sensor can reliably contact the user.

If the biological information detection unit 200 includes a plurality of contact members 210, or if a plurality of biological information detection units 200 is provided, at least one of a size, a shape, and a material for each contact member 210 of the biological information detection unit 200 may be set depending on the region in contact with the user. For example, if the contact member 210 is brought into contact with a submental area of the user, the contact member 210 with a narrow contact area can be used so that the contact member 210 is locally brought into contact with the submental area. If the contact member 210 is brought into contact with the cheek area of the user, the contact member 210 with a wide contact area can be used so as to prevent the cheek area from being deformed to a large extent. The area of the contact member 210 for the submental area can be set to be different from the area of the contact member 210 for the cheek area. In other words, the size of the contact member 210 can be set depending on the region where the biological information detection unit 200 is placed, or the region where the contact member 210 contacts.

The shape and material for the contact member 210 may be set depending on the region where the contact member 210 contacts.

The posture control member 220 is a member that controls at least one of a contact position and a contact posture so that at least one of the position and the posture of the contact member 210 can be appropriately set. In this case, appropriately setting the position and the posture means that a condition (wearing condition) for favorably obtaining biological information about the user is satisfied. The posture control member 220 controls at least one of the position and the posture of the contact member 210 so as to satisfy a predetermined wearing condition. The wearing condition indicates that the contact member 210 has such a positional relationship that the contact member closely adheres to the skin of a region to be measured of the user. Examples of a state where the wearing condition is not satisfied include a state where the contact member 210 is not in close contact with the skin of the user and kept afloat. This state also includes a state where a part of the contact member 210 is kept afloat. It may also be determined that the wearing condition is not satisfied in a case where the contact member 210 is in close contact with the skin at the time of wearing the device, but floats when the user performs an operation. However, the content of the wearing condition varies depending on the type of the sensor to be used.

The posture control member 220 is passively controlled so as to be adapted to the wearing position and movement of the user along with the wearing position and movement of the user. In other words, the posture control member 220 is deformed along with the wearing position and movement of the user. Further, the posture control member 220 may actively control the wearing position and movement of the user. For example, the posture control member 220 may include an actuator to change the shape of the posture control member 220, and may be dynamically deformed along with the wearing position and movement of the user.

The posture control member 220 according to the present embodiment is located between the contact member 210 and the biasing member 113 (second member). The posture control member 220 includes an elastic member 221 to be deformed by an external force received by the contact member 210 from the user. As a material for the elastic member 221, a silicone gel, synthetic rubber, sponge, urethane, bellows, or the like may be mainly used.

As the elastic member 221, for example, a sponge with a cellular structure can be used. The sponge with the cellular structure is formed of a plurality of air bubbles connected together. Accordingly, gas can pass through the sponge with the cellular structure. When the biological information detection unit 200 is worn on the user, the elastic member 221 is compressed, and when the biological information detection unit 200 is detached from the user, the elastic member 221 contains gas and is restored.

As the elastic member 221, for example, bellows can be used. The bellows are located between the contact member 210 and the biasing member 113 (second member). When the biological information detection unit 200 is worn on the user, the bellows are contracted, and when the biological information detection unit 200 is detached from the user, the bellows are restored.

As the elastic member 221, a spiral spring or the like made of metal or resin can be used. The material of the elastic member 221 is not limited to the above-described materials, and any material may be used as long as the material has elasticity.

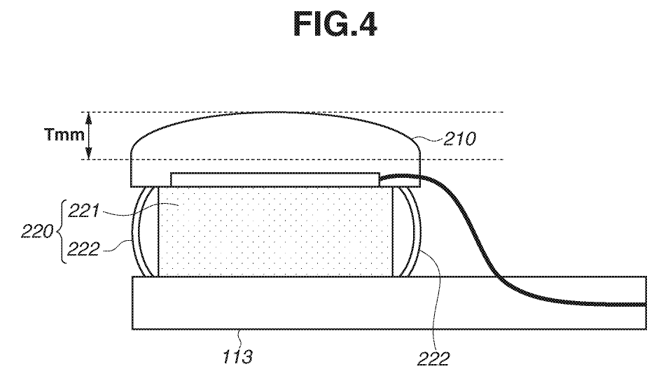

FIGS. 4 to 9 illustrate configurations of the elastic member 221 and a deformation limiting member 222 in the biological information detection unit 200.

FIG. 4 illustrates a deformation amount of the elastic member 221 in the biological information detection unit 200. The elastic member 221 has such a deformation function that the elastic member can be expanded and contracted by the amount corresponding to a predetermined expansion/contraction width (Tmm: 5 mm, for example). In this case, the elastic member 221 can be expanded and contracted by Tmm in the vertical direction. The expansion/contraction width of the elastic member 221 can be arbitrarily set by changing the material of the elastic member 221. The biological information detection unit 200 may detect the deformation amount of the elastic member 221 as biological information.

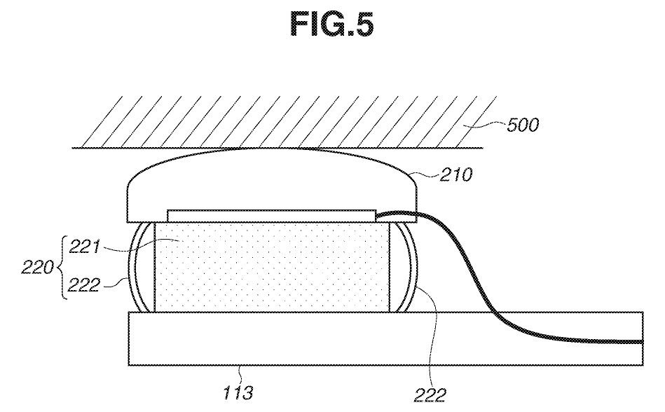

FIG. 5 illustrates a configuration in which a user (skin surface) 500 contacts an upper surface (contact member 210) of the biological information detection unit 200. In this case, the elastic member 221 is not deformed.

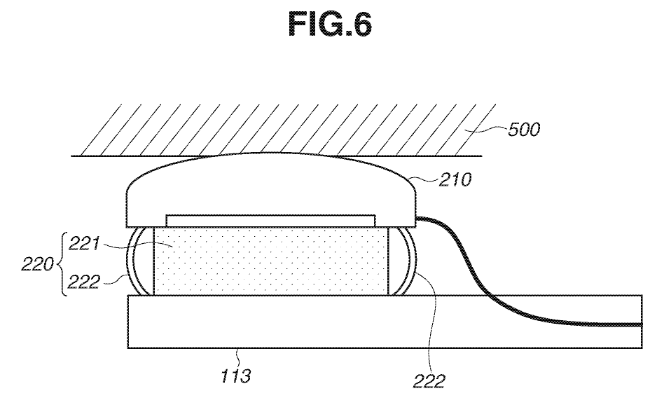

FIG. 6 illustrates a configuration in which the user (skin surface) 500 contacts the upper surface (contact member 210) of the biological information detection unit 200 and the elastic member 221 in the biological information detection unit 200 is compressed downward. The elastic member 221 has such a deformation function that the elastic member can be expanded and contracted by the amount corresponding to the predetermined expansion/contraction width. Accordingly, as illustrated in FIG. 6, the elastic member 221 is deformed downward. A reaction force from the deformed elastic member 221 causes the upper surface (contact member 210) of the biological information detection unit 200 to be pushed out upward, thereby making it possible to bring the biological information detection unit 200 into close contact with the user (skin surface) 500.

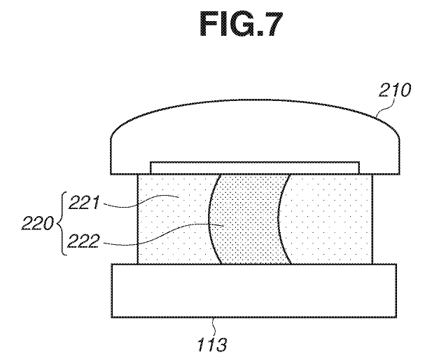

As illustrated in FIG. 3, the posture control member 220 includes the deformation limiting member 222 that limits deformation in the elastic member 221. FIG. 7 illustrates the biological information detection unit 200 illustrated in FIG. 3 as viewed from the left side. The deformation limiting member 222 is connected to each of the contact member 210 and the biasing member 113 (second member) and is placed.

The deformation limiting member 222 is composed of a member with predetermined flexibility and is not stretched beyond a predetermined length. The predetermined length corresponds to a length in the vertical direction in the drawing. The deformation limiting member 222 is a band-like member. For example, fabrics (cloth), a metallic thin film, or the like can be used. The deformation limiting member 222 may be a linear member. For example, a wire or a string member can be used.

As illustrated in FIGS. 3 and 7, the deformation limiting member 222 is connected to each of the contact member 210 and the biasing member 113 (second member) and is placed. The deformation limiting member 222 is placed in the vicinity of the elastic member 221. FIG. 3 illustrates an example where a plurality of deformation limiting members 222 is provided and is formed of a plurality of members. Each of the plurality of deformation limiting members 222 is connected to, for example, the contact member 210 and the biasing member 113 (second member) at two locations. The plurality of deformation limiting members 222 is a pair of deformation limiting members, and each of the deformation limiting members 222 is connected to the contact member 210 and the biasing member 113 (second member). As illustrated in FIGS. 3 and 7, the right-end deformation limiting member 222 and the left-end deformation limiting member 222 form a pair of deformation limiting members. The right-end deformation limiting member 222 and the left-end deformation limiting member 222 are each connected to the contact member 210 and the biasing member 113 (second member).

Each deformation limiting member 222 is connected to the contact member 210 and the biasing member 113 (second member) at two locations, but instead may be connected at four or more locations.

If the deformation limiting member 222 is a band-like member as illustrated in FIG. 7, an upper end and a lower end of the band-like member are each connected to the contact member 210 and the biasing member 113 (second member). The deformation limiting member 222 is connected to each of the contact member 210 and the biasing member 113 (second member) via an upper-end joining area of the band-like member and a lower-end joining area of the band-like member. The upper-end joining area of the deformation limiting member 222 has a predetermined width, and the deformation limiting member 222 is connected to the contact member 210. With this configuration, the deformation limiting member 222 can apply a force to the contact member 210 via the upper-end joining area. Similarly, the lower-end joining area in the deformation limiting member 222 has a predetermined width, and the deformation limiting member 222 is connected to the biasing member 113 (second member). With this configuration, the deformation limiting member 222 can apply a force to the biasing member 113 (second member) via the lower-end joining area.

If the deformation limiting member 222 is a band-like member having a width more than or equal to the predetermined width, the deformation limiting member 222 can cover a part of the elastic member 221. The deformation limiting member 222 can also cover the surrounding area of the elastic member 221. Accordingly, the elastic member 221 can be hidden by the deformation limiting member 222.

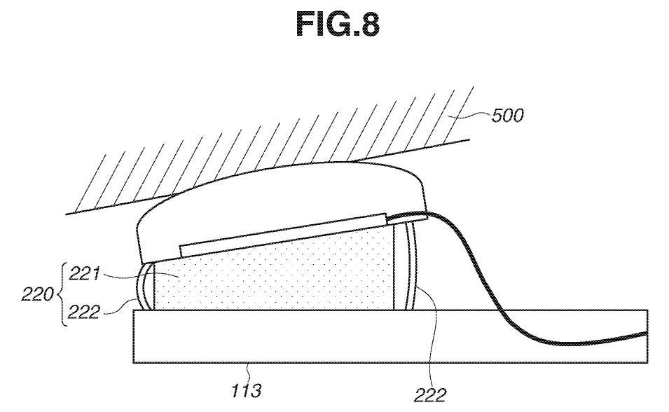

FIG. 8 is a figure based on FIG. 3, and illustrates a configuration in which the user (skin surface) 500 obliquely contacts the upper surface (contact member 210) of the biological information detection unit 200 and the biological information detection unit 200 is compressed obliquely downward.

The elastic member 221 is bent obliquely. In this case, FIG. 8 illustrates a configuration in which the left-side elastic member 221 is bent and compressed and the right-side elastic member 221 is bent and stretched.

As illustrated in FIG. 8, the right-end deformation limiting member 222 is stretched together with the stretched elastic member 221. Since the right-end deformation limiting member 222 is a member that is not stretched beyond the predetermined length, when the right-end deformation limiting member 222 reaches the predetermined length, the right-end deformation limiting member 222 can suppress deformation of the stretched elastic member 221. The deformation limiting member 222 can suppress deformation of the elastic member 221 that can be arbitrarily deformed. Accordingly, the contact position and the contact posture of the biological information detection unit 200 with respect to the user (skin surface) 500 can be limited. The left-end deformation limiting member 222 may suppress deformation of the compressed elastic member 221.

FIG. 8 illustrates a configuration in which the left-side elastic member 221 is bent and compressed and the right-side elastic member 221 is bent and stretched. Also, in a configuration in which the right-side elastic member 221 is bent and compressed and the left-side elastic member 221 is bent and stretched, the same advantageous effect can be obtained. In this case, the left-end deformation limiting member 222 is stretched together with the stretched elastic member 221. Since the left-end deformation limiting member 222 is a member that is not stretched beyond the predetermined length, when the left-end deformation limiting member 222 reaches the predetermined length, the left-end deformation limiting member 222 can suppress deformation of the stretched elastic member 221.

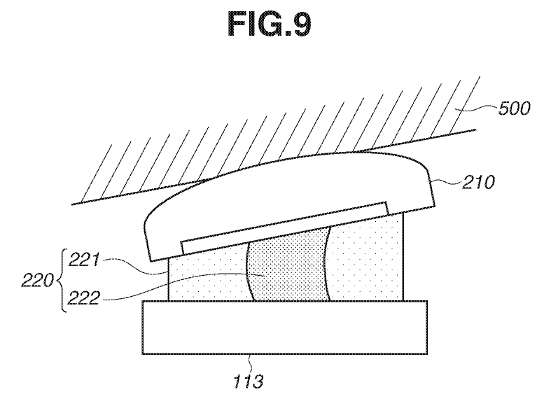

FIG. 9 is a figure based on FIG. 7, and illustrates a configuration in which the user (skin surface) 500 obliquely contacts the upper surface (contact member 210) of the biological information detection unit 200 and the biological information detection unit 200 is compressed obliquely downward.

The elastic member 221 is bent obliquely. In this case, FIG. 9 illustrates a configuration in which the left-side elastic member 221 is bent and compressed and the right-side elastic member 221 is bent and stretched.

As illustrated in FIG. 9, the deformation limiting member 222 is deformed together with the deformed elastic member 221. Since the right end portion of the deformation limiting member 222 is not stretched beyond the predetermined length, when the deformation limiting member 222 reaches the predetermined length, the right end portion (one end) of the deformation limiting member 222 can suppress deformation on the right side of the expanded elastic member 221. The deformation limiting member 222 can suppress deformation of the elastic member 221 that can be arbitrarily deformed. Accordingly, the contact position and the contact posture of the biological information detection unit 200 with respect to the user (skin surface) 500 can be limited. The left end portion (other end) of the deformation limiting member 222 may suppress deformation of the compressed elastic member 221.

FIG. 9 illustrates a configuration in which the left-side elastic member 221 is bent and compressed and the right-side elastic member 221 is bent and stretched. Also, in a configuration in which the right-side elastic member 221 is bent and compressed and the left-side elastic member 221 is bent and stretched, the same advantageous effect can be obtained. In this case, since the left end portion (other end) of the deformation limiting member 222 is not stretched beyond the predetermined length, when the deformation limiting member 222 reaches the predetermined length, the left end portion (other end) of the deformation limiting member 222 can suppress deformation on the left side of the expanded elastic member 221. The right end portion (one end) of the deformation limiting member 222 may suppress deformation of the compressed elastic member 221.

The posture control member 220 and the sensor 230 need not necessarily be separated from each other, and the posture control member 220 may function as a part of the sensor 230. For example, the posture control member 220 may be combined with an optical sensor or a strain gauge to detect the deformation amount of the posture control member 220, and the detected deformation amount may be used as biological information.

According to the present embodiment described above, the information conversion system that outputs character information or audio information converted from biological information about the user includes the first member (frame member 110 (first member)) to be fixed to the user. The information conversion system further includes the biological information detection unit 200 configured to bring the contact member 210 into contact with the user to detect biological information. The information conversion system further includes the second member (biasing member 113 (second member)) that is placed on the first member (frame member 110 (first member)) and biases the biological information detection unit 200 in a direction in which the biological information detection unit contacts the user. The biological information detection unit 200 includes the posture control member 220 that controls the posture of the contact member 210.

Consequently, the sensor in the information conversion system or a biological information detection apparatus that converts biological information into character information can be repeatedly worn.

### Modified Example 1

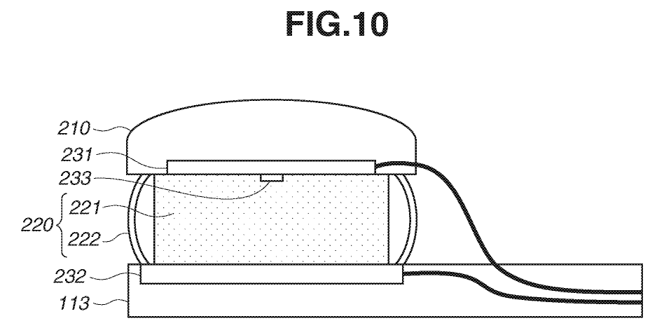

Next, Modified Example 1 of the present disclosure will be described. FIG. 10 illustrates a configuration of the biological information detection unit 200 according to Modified Example 1. In the present modified example, a contact type sensor unit is used as the biological information detection unit 200, and a magnetic sensor, an acceleration sensor, and an angular velocity sensor are used.

The biological information detection unit 200 according to the present modified example includes the contact member 210, the posture control member 220, a circuit board 231 on which a magnetic sensor integrated circuit (IC) is mounted, and a circuit board 232 on which an inertial sensor IC is mounted. The inertial sensor IC incorporates a three-axis acceleration detection unit and a three-axis angular velocity detection unit.

A permanent magnet 233 is fixed to the posture control member 220 side of the contact member 210. The magnetic sensor IC on the circuit board 231 detects a displacement relative to the permanent magnet 233. The magnetic sensor IC is an element having four magnetic detection points. Alternatively, a three-axis geomagnetic sensor configured to detect a magnetic field vector direction may be used. A three-dimensional displacement of the permanent magnet 233 is detected by a subsequent-stage signal processing circuit (not illustrated) using an output from the magnetic sensor IC, and an external force is calculated based on the detected displacement amount.

The three-axis acceleration detection unit and the three-axis angular velocity detection unit on the circuit board 232 are an acceleration sensor and an angular velocity sensor, respectively. A three-axis acceleration and a three-axis angular velocity in the biological information detection unit 200 are detected. Examples of the type of the acceleration sensor in the biological information detection unit 200 include a piezoelectric type, a piezoresistive type, and a capacitance type. Examples of the type of the angular velocity sensor include a piezoelectric type and a capacitance type.

The biological information detection unit 200 can detect biological information using a displacement relative to the permanent magnet 233, an acceleration, and an angular velocity. The wearing determination unit 121 determines whether the wearing condition is satisfied based on whether the biological information detected in the magnetic sensor, the acceleration sensor, and the angular velocity sensor has been appropriately detected.

Specifically, the wearing determination unit 121 determines whether the wearing condition is satisfied using the displacement relative to the permanent magnet 233, the acceleration, and the angular velocity detected in the magnetic sensor, the acceleration sensor, and the angular velocity sensor, respectively. For example, if the displacement relative to the permanent magnet 233 detected in the magnetic sensor falls within a predetermined displacement, the wearing determination unit 121 determines that the wearing condition is satisfied. If the acceleration detected in the acceleration sensor is less than or equal to a predetermined acceleration, the wearing determination unit 121 determines that the wearing condition is satisfied. If the angular velocity detected in the angular velocity sensor is less than or equal to a predetermined angular velocity, the wearing determination unit 121 determines that the wearing condition is satisfied.

### Modified Example 2

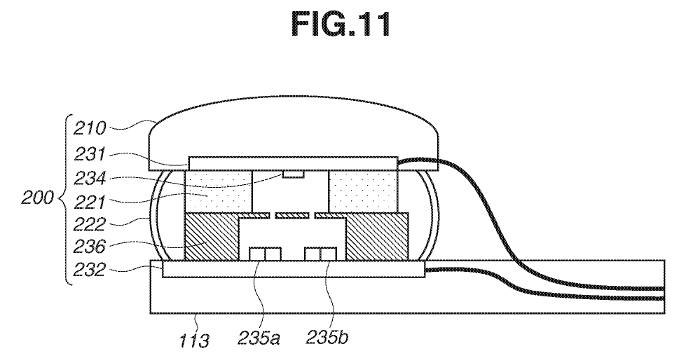

Next, Modified Example 2 of the present disclosure will be described. Descriptions of parts that are common to those of Modified Example 1 are omitted. FIG. 11 illustrate a configuration of the biological information detection unit 200 according to Modified Example 2. In the present modified example, an optical tactile sensor unit is used as the biological information detection unit 200.

The biological information detection unit 200 according to the present embodiment includes a circuit board on which two-segmented photodiodes 235a and 235b are mounted, and a light-emitting element 234 is further mounted on the circuit board 231 on which the inertial sensor IC is mounted. In this case, the light-emitting element 234 is placed on the contact member 210. The two-segmented photodiodes 235a and 235b are placed on the biasing member 113 (second member). A hollow space is formed between the light-emitting element 234 and the divided photodiodes 235a and 235b. In other words, the elastic member 221 is not present between the light-emitting element 234 and the divided photodiodes 235a and 235b.

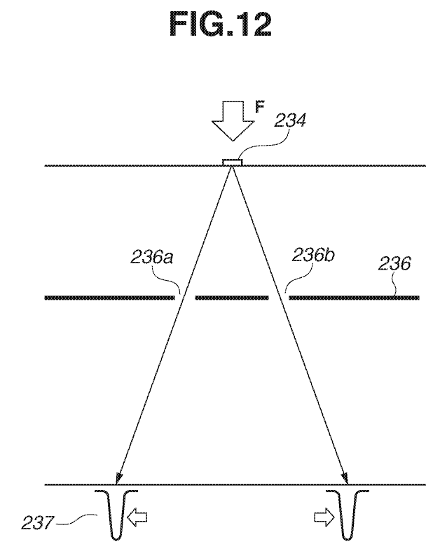

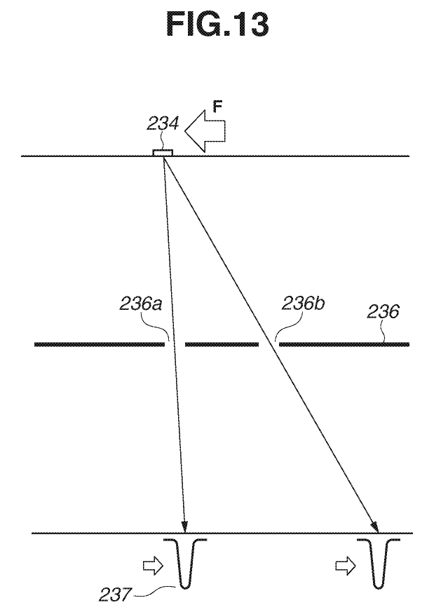

The two-segmented photodiodes 235a and 235b are light-receiving elements each having a two-segmented light-receiving surface. As illustrated in FIGS. 12 and 13, an opening member 236 includes an opening 236a and an opening 236b that are formed in a light path between the light-emitting element 234 and the two-segmented photodiodes 235a and 235b.

A three-dimensional displacement of the light-emitting element 234 is detected by the subsequent-stage signal processing circuit (not illustrated) using outputs from the two-segmented photodiodes 235a and 235b, and an external force is calculated based on the detected displacement amount.

A procedure for detecting the amount of movement of the light-emitting element 234 due to an external force and calculating the external force will now be described. FIG. 12 illustrates a state of movement of the light-emitting element 234 when an external force F in the vertical direction is received and a state of movement of a projected spot 237. A distance between a spot pair formed by a single light-emitting element 234 varies depending on a ratio between a distance between the light-emitting element 234 and each of the opening 236a and the opening 236b and a distance between a light-receiving IC surface and each of the opening 236a and the opening 236b. Upon receiving a pressing force in a planar direction, the light-emitting element 234 moves downward, so that the distance between the light-emitting element 234 and each of the opening 236a and the opening 236b decreases. As a result, the center-to-center distance of the spot pair increases. The external force in the vertical direction can be estimated by calculating the distance between center position coordinates of the spot pair.

FIG. 13 illustrates a state of a movement of the light-emitting element 234 when the external force F in a shearing direction is received, and a state of a movement of the projected spot 237. When the elastic member 221 receives the external force in the shearing direction via the contact member 210, the light-emitting element 234 moves in an in-plane direction. As a result, the central position of the spot pair moves in a direction opposite to that of the external force. The external force in the shearing direction can be estimated by calculating an average value of the center position coordinates of the spot pair.

### Modified Example 3

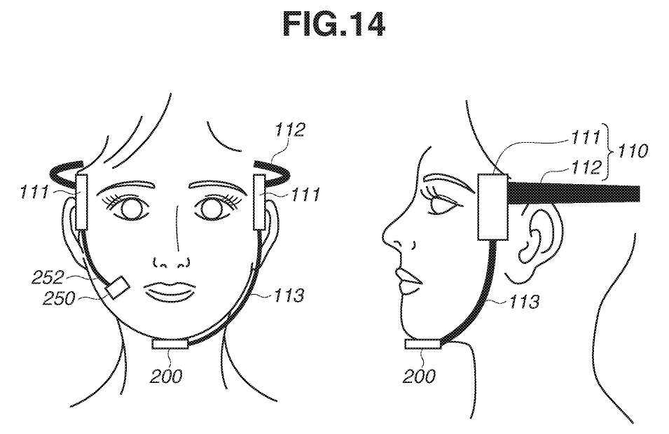

Next, Modified Example 3 of the present disclosure will be described. FIG. 14 illustrates an appearance of an information conversion system according to Modified Example 3.

The biasing member 113 (second member) that biases the biological information detection unit 200 is provided on (extended from) the frame member 110 (first member). Further, a biasing member 252 that biases a biological information detection unit 250 is placed on (extended from) the frame member 110 (first member).

The biological information detection unit 200 is placed on a submental area of the user and the biological information detection unit 250 is placed on the cheek of the user.

The biasing member 252 is a biasing member that biases the biological information detection unit 250 in the direction in which the biological information detection unit contacts the user. The biasing member 252 has elasticity in the direction in which the biasing member contacts the user so as to maintain the biasing force. Thus, the biasing member 252 enables the biological information detection unit 250 to be pressed against the user.

The biasing member 252 is connected to the biological information detection unit 250. The biological information detection unit 250 is a component that moves along with the oral movement of the user. Accordingly, the biasing member 252 may have elasticity so that the biological information detection unit 250 can be pressed against the user and the biasing member is deformed along with the oral movement. The rigidity of the biasing member 252 is set to a third predetermined value or higher so that the biological information detection unit 250 can be biased in the direction in which the biological information detection unit contacts the user. The rigidity of the biasing member 252 is lower than the rigidity of the connecting portion 112 (frame member 110 (first member)). The rigidity of the biasing member 252 is higher than the rigidity of the biasing member 113 (second member).

The rigidity of the connecting portion 112 (frame member 110 (first member)) is set to the second predetermined value or higher. The rigidity is at least one of bending rigidity and shear rigidity. Accordingly, the third predetermined value for the rigidity of the biasing member 252 is lower than the second predetermined value for the rigidity of the connecting portion 112 (frame member 110 (first member)).

The biasing member 252 can be desirably configured to adjust the biasing force so as not to inhibit the oral movement of the user. Like the connecting portion 112, the biasing member 252 includes an adjustment mechanism, which enables adjustment of the biasing force of the biological information detection unit 250 connected to the biasing member 252. Examples of the adjustment mechanism include the slide mechanism (expansion/contraction mechanism) in the biasing member 252.

### Modified Example 4

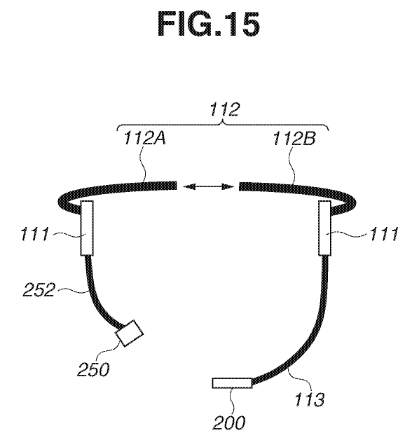

Next, Modified Example 4 of the present disclosure will be described. FIG. 15 illustrates an appearance of an information conversion system according to Modified Example 4. Modified Example 4 differs from Modified Example 3 in that the connecting portion 112 that constitutes the frame member 110 (first member) is composed of a connecting portion 112A and a connecting portion 112B. In a case where the user wears the device, the user can wear the device from the left side and the right side of the user's head so as to prevent the biasing members 113 and 252 from interfering with wearing the device. The connecting portion 112A and connecting portion 112B can be connected with, for example, a magnet or buckle, but instead may be connected by various methods. The connecting portion 112A and the connecting portion 112B may be provided with an adjustment mechanism that is formed of a plurality of tubular members and is configured to adjust the length. This configuration enables the user to easily take on or take off the device. Further, the device can be accommodated in a compact manner.

The device may be powered on when the connecting portion 112A and the connecting portion 112B are connected. The wireless communication between the information processing unit 120 and the sensor 230 and the wireless connection among the communication unit 130, the external apparatus 300, the display unit 310, and the audio information output unit 320 may be started with the connection between the connecting portion 112A and the connecting portion 112B as a trigger. Further, the wearing determination may be performed and estimation may be started with the connection between the connecting portion 112A and the connecting portion 112B as a trigger. The device may be powered off when the connecting portion 112A and the connecting portion 112B are disconnected. This configuration enables the user to use the information conversion system more simply. Moreover, wasteful power consumption can be avoided.

### Modified Example 5

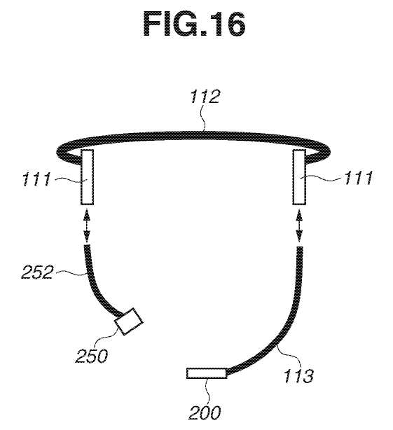

Next, Modified Example 5 of the present disclosure will be described. FIG. 16 illustrates an appearance of an information conversion system according to Modified Example 5. Modified Example 5 differs from Modified Example 3 in that the sandwiching portions 111 that constitute the frame member 110 (first member) and the biasing members 113 and 252 (second member) are separate members. The user can wear the device in a state where the biasing members 113 and 252 are detached from the first member 110. After the first member is worn, the biasing members 113 and 252 are connected to the sandwiching portions 111, thereby enabling the user to easily wear the device. The biasing members 113 and 252 and the sandwiching portions 111 can be connected with, for example, a magnet. The biasing members 113 and 252 may be provided with an adjustment mechanism that is formed of a plurality of members and is configured to adjust the length. This configuration enables the device to be accommodated in a compact manner.

Like in Modified Example 4, power-on and power-off of the device may be switched with the connection and disconnection between the biasing members 113 and 252 and the sandwiching portions 111 as a trigger. Further, the wireless communication between the information processing unit 120 and the sensor 230 and the wireless communication among the communication unit 130, the external apparatus 300, the display unit 310, and the audio information output unit 320 may be started and ended with the connection and disconnection between the biasing members 113 and 252 and the sandwiching portions 111 as a trigger. Further, the wearing determination and estimation may be started with the connection as a trigger. This configuration enables the user to use the information conversion system more simply. Moreover, wasteful power consumption can be avoided.

### Modified Example 6

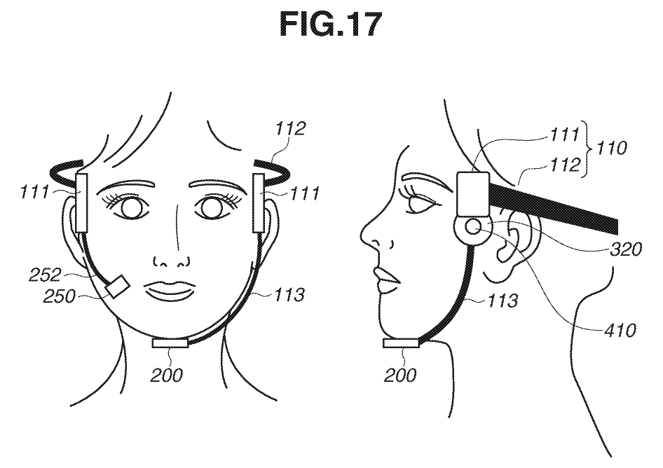

Next, Modified Example 6 of the present disclosure will be described. FIG. 17 illustrates an appearance of an information conversion system according to Modified Example 6. Modified Example 6 differs from Modified Example 5 in that the audio output unit 320 is placed on the sandwiching portions 111 or the like. Examples of the audio output unit 320 include bone-conduction earphones, canal-type earphones, and headphones. The user can check audio information output from the information processing unit 120 via the audio output unit 320. The position where the audio output unit 320 is placed is not limited to the sandwiching portions 111, but instead may be placed on the connecting portion 112 or the biological information detection unit 200, or may be placed in the ears. The audio output unit may be placed at any location as long as the audio information output from the information processing unit 120 can be checked. This configuration enables the user to easily check whether the oral movement of the user can be converted into intended audio information. The audio output unit 320 may output not only the audio information estimated based on the biological information about the user, but also music, voice of a telephone conversation partner, or the like.

In the present modified example, an audio input unit 410 is placed on the sandwiching portions 111 or the like. The audio input unit 410 is, for example, a microphone. The position where the audio input unit 410 is placed is not limited to on the sandwiching portions 111, and may be placed on the connecting portion 112 or the biological information detection unit 200. The audio input unit may be placed at any location as long as audio information about the user can be obtained. The audio information input to the audio input unit 410 is sent to the information processing unit 120.

The information processing unit 120 estimates the corresponding character information or audio information based on the biological information and the audio information obtained by the biological information detection unit 200 and the biological information detection unit 250, and outputs the corresponding character information or audio information. Thus, the information processing unit 120 can increase the estimation accuracy based on the biological information and the audio information. Further, the information processing unit 120 may estimate and output the corresponding character information based only on audio information, and may output the audio information from which noise is removed.

The type of information to be input to the information processing unit 120, that is, only audio information, only biological information, or audio information and biological information, may be switched by a user operation, or may be automatically switched depending on the connection state between the biasing members 113 and 252 and the sandwiching portions 111. Specifically, in a state where the biasing members 113 and 252 and the sandwiching portions 111 are not connected, only audio information may be input to the information processing unit 120. In a state where the biasing members 113 and 252 and the sandwiching portions 111 are connected, audio information and biological information may be input to the information processing unit 120. The information to be input may be automatically switched. Even in the state where the biasing members 113 and 252 and the sandwiching portions 111 are connected, if it is determined that the audio information includes no audio information about the user, the information processing unit 120 may automatically switch the information to be input so as to estimate and output the corresponding character information or audio information based only on biological information. Also, in the state where the biasing members 113 and 252 and the sandwiching portions 111 are connected, only audio information may be input to the information processing unit 120.

This configuration enables the user to use the information conversion system with an optimum hardware configuration depending on usage conditions.

### Modified Example 7

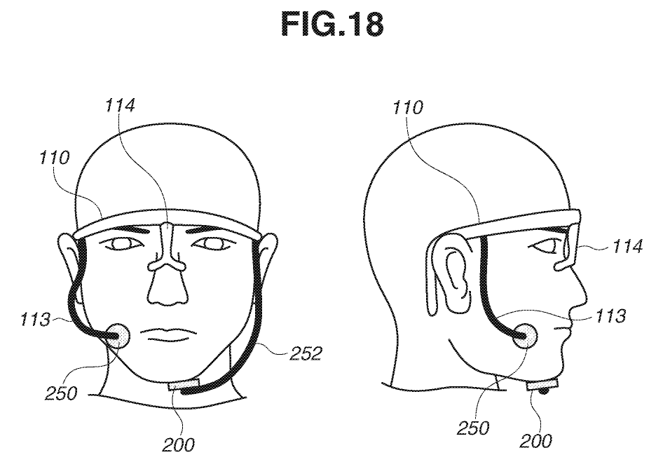

Next, Modified Example 7 of the present disclosure will be described. The present modified example is an example where the frame member 110 according to Modified Example 3 lies across the front side of the face. FIG. 18 illustrates an appearance of an information conversion system according to Modified Example 7.

The frame member 110 is provided with a nose pad member 114. A leading end of the nose pad member 114 is divided into two sections, and the nose pad member contacts the upper portion of the nose in the worn state. In this case, the frame member 110 may function as glasses, or may be a part of a glasses-type image output device.

The biasing member 113 (second member) that biases the biological information detection unit 200 is placed on (extended from) the frame member 110 (first member). Further, the biasing member 252 that biases the biological information detection unit 250 is placed on (extended from) the frame member 110 (first member). The biological information detection unit 200 is placed on a submental area of the user and the biological information detection unit 250 is placed on the cheek of the user. The biasing member 252 is a biasing member that biases the biological information detection unit 250 in the direction in which the biological information detection unit contacts the user. The biasing member 252 has elasticity in the direction in which the biasing member contacts the user so as to maintain the biasing force. Thus, the biasing member 252 can press the biological information detection unit 250 against the user.

According to the present modified example as described above, the device can be worn from the front side of the face, which makes it possible for the user to easily wear the device without disturbing the hairstyle. Further, fixing portions can be supported by the ears and nose, which enables the frame member 110 to be stably fixed to the head even when forces in various directions are received due to opening and closing of the mouth through the biasing member 113. This leads to an improvement in the accuracy of obtaining biological information.

### Modified Example 8

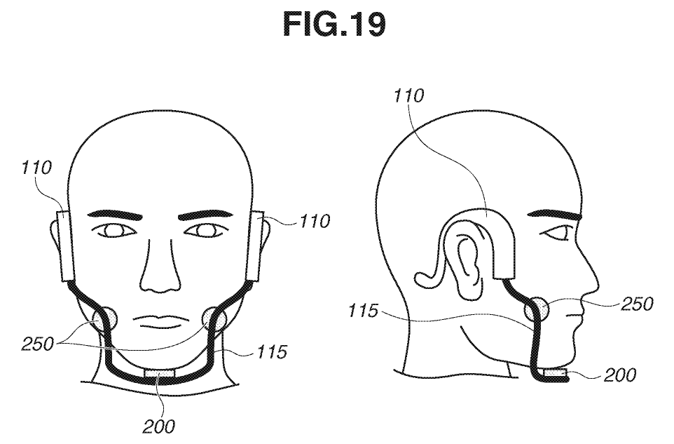

Next, Modified Example 8 of the present disclosure will be described. The present modified example is an example in which the frame member 110 according to Modified Example 3 is divided into two right and left members. FIG. 19 illustrates an appearance of an information conversion system according to Modified Example 8.

The two frame members 110 are connected via a biasing member 115. The biological information detection unit 200 is placed on a submental area of the user, and two biological information detection units 250 are placed on the cheeks of the user. In this case, the biological information detection units 250 are placed on the right and left cheeks, and may include a dummy pad having no detection function. The right and left frame members 110 are put on the ears and thus fixed to the head, and the elasticity of the biasing member 115 enables the biological information detection unit 200 and the biological information detection unit 250 to be pressed against the submental area and the cheeks. In this case, a force for sandwiching the cheeks between the two biological information detection units 250 acts, which makes it possible to stabilize the biasing force and prevents a wearing failure such as floating.

According to the present modified example as described above, the device can be worn from the front side of the face, which makes it possible for the user to easily wear the device without disturbing the hairstyle. In the configuration in which the cheeks are sandwiched between the detection units, the advantageous effect of preventing a wearing failure can be obtained.

### Modified Example 9

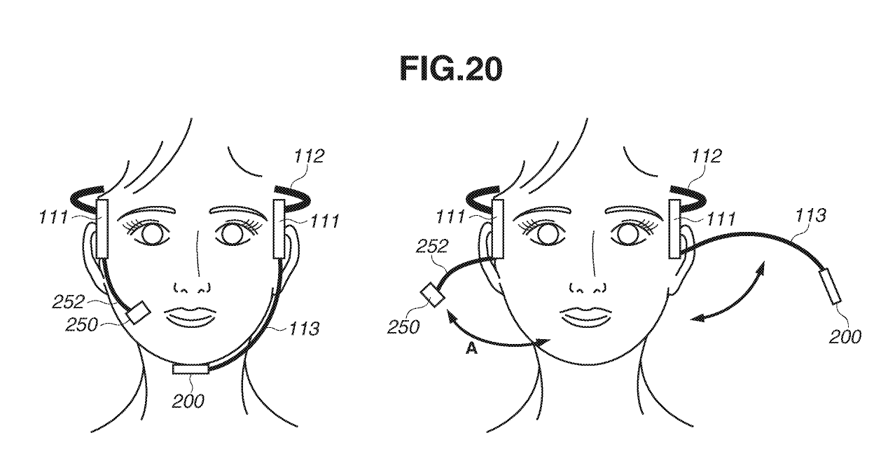

Next, Modified Example 9 of the present disclosure will be described. FIG. 20 illustrates an appearance of an information conversion system according to Modified Example 9. Modified Example 9 differs from Modified Example 3 in that a mechanism for rotating the biasing members 113 and 252 (second member) in the horizontal direction with respect to the sandwiching portions 111 that constitute the frame member 110 (first member) is provided.

This configuration enables the user to easily wear the device. The biasing members 113 and 252 may include a rotation mechanism with a rotation axis in the longitudinal direction of each biasing member. This configuration enables the device to be accommodated in a more compact manner. This rotation mechanism enables wearing of the device that is more adapted to the shape of the user's face.

The biasing members 113 and 252 may have a function for turning on the power supply of the device when a predetermined wearing position (position where the sensors 200 and 250 contact the skin) is reached, and turning off the power supply when a detachment position is reached. Further, power-on and power-off may be fed back to the user as sound through bone-conduction earphones incorporated in the biasing member 113.

This configuration enables the user to use the information conversion system more simply. Moreover, wasteful power consumption can be avoided.

### Modified Example 10

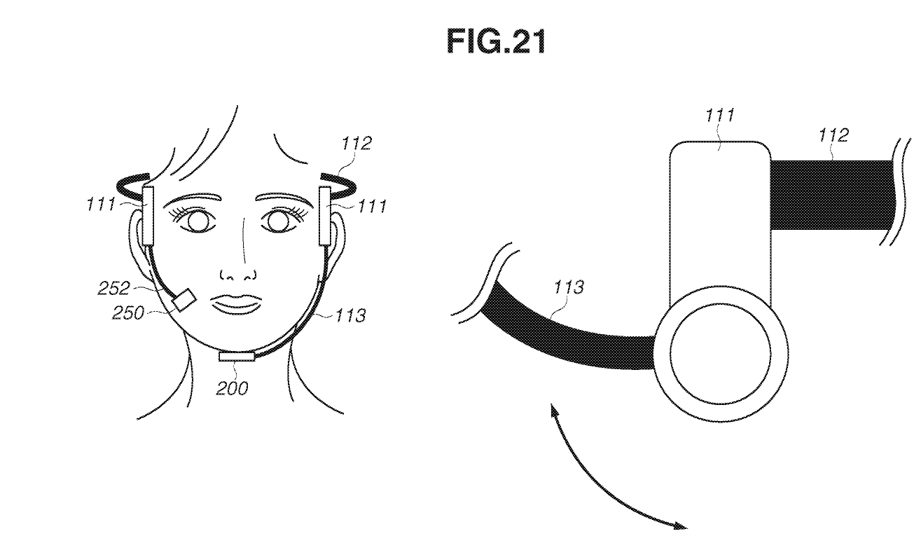

Next, Modified Example 10 of the present disclosure will be described. FIG. 21 illustrates an appearance of an information conversion system according to Modified Example 10. Modified Example 10 differs from Modified Example 3 in that a mechanism for rotating the biasing members 113 and 252 (second member) in the vertical direction with respect to the sandwiching portions 111 that constitute the frame member 110 (first member) is provided.

This configuration enables the user to easily wear the device. Further, the device can be accommodated in a more compact manner.

The biasing members 113 and 252 may have a function for turning on the power supply of the device when the predetermined wearing position (position where the sensors 200 and 250 contact the skin) is reached, and turning off the power supply when the detachment position is reached. Further, power-on and power-off may be fed back to the user as sound through bone-conduction earphones incorporated in the biasing member 113.

This configuration enables the user to use the information conversion system more simply. Moreover, wasteful power consumption can be avoided.

As described above, the present embodiment includes the first member (frame member 110 (first member)) to be fixed to the user, and the biological information detection unit 200 that brings the contact member 210 into contact with the user to detect biological information. The present embodiment further includes the second member (biasing member 113 (second member)) that is placed on the first member (frame member 110 (first member)) and biases the biological information detection unit 200 in the direction in which the biological information detection unit contacts the user. The present embodiment further includes the third member (biasing member 252) that is placed on the first member (frame member 110 (first member)) and biases the biological information detection unit 250 in the direction in which the biological information detection unit contacts the user.

The above-described embodiments of the present disclosure are merely examples of substantiation in implementing the present disclosure, and the technical scope of the present disclosure should not be construed to be limited by the embodiments. That is, the present disclosure can be implemented in various forms without departing from the technical idea thereof or the principal features thereof.

The present disclosure is not limited to the above embodiments and various changes and modifications can be made within the spirit and scope of the present disclosure. Therefore, to apprise the public of the scope of the present disclosure, the following claims are made.

According to the present disclosure, an information conversion system or a biological information detection apparatus that converts biological information into character information includes a sensor that can be repeatedly worn.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An information conversion system that outputs character information or audio information converted from biological information about a user, the information conversion system comprising:
a first member to be fixed to the user;
a biological information detection unit configured to bring a contact member into contact with the user to detect biological information; and
a second member placed on the first member and configured to bias the biological information detection unit in a direction in which the biological information detection unit contacts the user,
wherein the biological information detection unit includes a posture control member configured to control a posture of the contact member.

2. The information conversion system according to claim 1, wherein the biological information detection unit is configured using a contact member configured to contact a skin surface of the user, the posture control member, and a sensor configured to detect the biological information about the user.

3. The information conversion system according to claim 1, wherein the biological information detection unit is placed at a leading end of the second member configured to bias the biological information detection unit in the direction in which the biological information detection unit contacts the user.

4. The information conversion system according to claim 1, wherein the posture control member controls at least one of a position and a posture of the contact member to satisfy a predetermined wearing condition.

5. The information conversion system according to claim 1, wherein the posture control member is located between the contact member and the second member and is connected to each of the contact member and the second member.

6. The information conversion system according to claim 1, wherein the posture control member includes an elastic member to be deformed by an external force received by the contact member.

7. The information conversion system according to claim 6, wherein the posture control member includes a deformation limiting member configured to limit deformation in the elastic member.

8. The information conversion system according to claim 7, wherein the deformation limiting member is a band-like member or a linear member.

9. The information conversion system according to claim 7, wherein a plurality of the deformation limiting members is provided and each of the plurality of deformation limiting members is connected to the contact member and the second member.

10. The information conversion system according to claim 1, wherein the first member has a shape to go around a head of the user and has elasticity for sandwiching the head of the user.

11. The information conversion system according to claim 1, wherein the first member or the second member includes an expansion/contraction mechanism.

12. The information conversion system according to claim 1, wherein the first member includes a rotation mechanism configured to support the second member and rotate the second member.

13. The information conversion system according to claim 1, wherein a plurality of the biological information detection units is provided and at least one of a size, a shape, and a material of the contact member is set depending on a region in contact with the user.

14. The information conversion system according to claim 6, wherein the biological information detection unit detects a deformation amount of the elastic member as the biological information.

15. The information conversion system according to claim 1, further comprising a wearing determination unit configured to determine whether a wearing condition for the biological information detection unit is satisfied based on whether the biological information detected by the biological information detection unit has been appropriately detected.

16. The information conversion system according to claim 15, further comprising a display unit configured to display a determination result in a case where the wearing determination unit determines that the wearing condition is not satisfied.

17. The information conversion system according to claim 1, wherein the biological information detection unit is at least one of a myopotential sensor, an acceleration sensor, an ultrasonic sensor, a tactile sensor, an optical sensor, and a pressure sensor.

18. The information conversion system according to claim 1, further comprising a main body unit configured to process the biological information obtained by the biological information detection unit,
wherein the main body unit is placed in the first member.

19. The information conversion system according to claim 1, further comprising an information processing unit configured to convert the biological information detected by the biological information detection unit into character information or audio information using a learned model generated by learning the biological information and the character information or the audio information in association with each other.

20. A biological information detection apparatus comprising:
a first member to be fixed to a user;
a biological information detection unit configured to bring a contact member into contact with the user to detect biological information; and
a second member placed on the first member and configured to bias the biological information detection unit in a direction in which the biological information detection unit contacts the user,
wherein the biological information detection unit includes a posture control member configured to control a posture of the contact member.
